(19) United States
(12) Patent Application Publication
(10) Pub. No.: US 2022/0051788 A1
(43) Pub. Date: Feb. 17, 2022

(54) METHODS AND SYSTEMS FOR DYNAMIC AUTOMATION OF QUALITY CONTROL AND INFORMATION MANAGEMENT FOR AN IN VITRO FERTILIZATION (IVF) LABORATORY

(71) Applicant: Fertility Guidance Technologies, Newport Beach, CA (US)

(72) Inventor: Carol Curchoe, Newport Beach, CA (US)

(21) Appl. No.: 17/401,209

(22) Filed: Aug. 12, 2021

Related U.S. Application Data
(60) Provisional application No. 63/066,698, filed on Aug. 17, 2020.

(51) Int. Cl. G16H 40/20; G16H 30/20

(52) U.S. Cl. CPC G16H 40/20; G16H 30/20

(57) ABSTRACT

Described herein are methods and systems for assessing competency of an in vitro fertilization (IVF) technician. A computing device selects an image of a biological entity associated with an IVF procedure. The computing device executes image analysis algorithms on the selected image to identify morphological characteristics of the biological entity. The computing device compares the identified morphological characteristics in the selected image with morphological characteristics of a biological entity in a reference image. The computing device generates a similarity score between the biological entity in the selected image and the biological entity in the reference image based upon the comparison, and determines an IVF procedure action for the biological entity in the selected image based upon the similarity score. The computing device requests input from a client computing device relating to the biological entity in the selected image, and determines whether the user input matches the IVF procedure action.

### RELATED APPLICATIONS

This application claims priority to U.S. Provisional Patent Application No. 63/066,698, filed on Aug. 17, 2020, the entirety of which is incorporated herein by reference.

### TECHNICAL FIELD

This application relates generally to methods and apparatuses, including computer program products, for dynamic automation of quality control and information management for an in vitro fertilization (IVF) laboratory.

### BACKGROUND

Generally, IVF is a type of assisted reproductive technology (ART) in which an egg is combined with sperm outside the body, in vitro (“in glass”), in order to fertilize the egg. The process involves monitoring and stimulating a woman's ovulatory process, removing an ovum or ova (egg or eggs) from the woman's ovaries and letting sperm fertilize them in a liquid in a laboratory. After the fertilized egg (zygote) undergoes embryo culture for a period of days, it is implanted in the same or another woman's uterus, with the intention of establishing a successful pregnancy. The IVF process is typically performed in a specialized medical facility called an IVF laboratory, which employs highly-trained personnel responsible for creating a patient's embryos, reporting their quality, status, and genetic results to both patients and physicians. IVF laboratory staff make dozens of critical clinical decisions for each patient's IVF cycle. For example, the staff are tasked with choosing the healthiest sperm to inseminate an egg with, whether to freeze or discard an embryo, and how best to carefully pluck the cells from the embryo during the surgical biopsy that will provide the genetic testing results.

Accredited IVF laboratories must document continuous monitoring of quality control and assurance for these and many other procedures. Infertility is one of the most regulated medical sub-specialties in the U.S. and worldwide. The U.S. Federal Fertility Clinic Success Rate and Certification Act of 1992 (FCSRCA) requires that “each ART program shall annually report . . . pregnancy success rates achieved by such program through each assisted reproductive technology.”

However, the time and documentation required to fulfill these and the many other regulations provided by the Centers for Disease Control (CDC), Food and Drug Administration (FDA), Occupational Safety and Health Administration (OSHA), Health Insurance Portability and Accountability Act (HIPAA), and Clinical Laboratory Improvement Amendments (CLIA), among others, takes valuable time from patient care, and errors in documentation can be very costly for an IVF clinic.

Also, competency of the medical professionals and personnel responsible for the IVF process at laboratories can be crucial to producing the best outcomes for patients. Embryology and andrology procedures are subjective, complex, and difficult to standardize. The lack of rigorously standardized laboratory protocols and strict quality control (QC) confounds even the best laboratories. Easily ensuring compliance with LQMS, CLIA, and WHO standards is an invaluable tool for clinics and laboratory directors. Accurate laboratory test results depend on staff being competent to perform a range of procedures.

For example, if a staff member is inadequately trained on particular equipment or does not capably handle and assess the viability of specimens, the IVF process can be unsuccessful. In the lab, competency survey tools are typically paper-based, meaning they can be lost or tampered with. Furthermore, testing materials, such as slides and CDs are re-used and the laboratory is expected to replace them every six months, inconveniencing lab managers and inviting ‘bias’ into the testing.

In addition, IVF is psychologically and emotionally stressful for patients and this has been proven to prevent a woman from attaining and maintaining a pregnancy. Many times, patents can feel disconnected from or out of control of the IVF process due to the lack of detailed information about their specific IVF process and status that is available for them to access.

Along with the above challenges, there are other significant challenges to implementing an artificial intelligence (AI) computing system in a meaningful way into a clinical IVF laboratory workflow. Implementation of AI at the actual point of care in a routine, easy, and automated fashion is nearly impossible for the majority of IVF labs that still use paper charts and do not take a single image of their patient's embryos, much less capturing videos. Additionally, data management solutions to augment patient demographics, clinical and lab key performance indicators (KPIs) and other relevant data streams (e.g., ultrasound images, preimplantation genetic testing (PGT) results, competency assessments) into a single dashboard are lacking.

Aside from the current lack of prospective data, there is another glaring flaw in all studies that use embryo images and video (analyzed by both AI and statistical models) to predict pregnancy outcome. The most common type of images used are blastocyst images taken before biopsy and cryopreservation, thawing, and transfer. The culture conditions of the lab, technical competency of the operator (both clinical and laboratory) and embryo quality and/or expansion post-thaw are not analyzed in models that use clinical pregnancy as an end-point, despite being dependent on them.

### SUMMARY

Therefore, what is needed are computerized methods and systems for dynamic automation of quality control and information management for an in vitro fertilization (IVF) laboratory, to overcome the aforementioned challenges. The techniques described herein advantageously provide an unprecedented level of access and oversight to the myriad of data and workflow processes required to operate an efficient and safe IVF laboratory, through the use of a networked arrangement of computing devices, data access portals, and software applications. The technology described herein take the pain out of managing dozens of parameters for automated regulatory reporting, simplifying the quality control workflow for IVF labs, saving time and money, all while simultaneously improving patient care by providing direct and timely access to hundreds of data points pertaining to each patient's IVF cycle.

As a result of these improvements, physicians can use the technology to make clinical care decisions, such as which embryo to transfer. Administrators can use the technology to track pregnancy and success rates for their clinic. Patients can use the technology to access a secure and HIPAA-compliant computerized portal to view their frozen embryo inventory, IVF cycle information, and direct access to physicians and laboratory staff. In addition, the methods and systems described herein beneficially provide patients a “visual tissue inventory” within the patient portal, giving them access to photos and videos of their developing embryos to help them feel more connected, involved, and in control, and allowing for instant updates every step along the way, reducing anxiety and stress.

Also, the techniques described herein enable seamless, software-based monitoring of staff competency assessments, tracking of key performance indicators, monitoring of culture conditions, and communicating with IVF laboratory staff via, e.g., a mobile application to assess competency, track staff related metrics, and view comparative data at the individual and clinic level. For example, the techniques provided herein offer distinct advantages over paper- and CD ROM-based legacy approaches: rapid data collection and real-time analysis and reporting, simple management and distribution of multi-media files, and push notifications to ensure timely testing.

Looking to the future, AI for reproduction must transcend packaging previously established pregnancy predictors in new machine learning algorithms. Another advantage offered by the methods and systems described herein is the use of prospective design, big data, standardized outcome measures, and external validation, integration of clinical and laboratory KPIs, plus patient demographics that help identify novel variables and hidden relationships that allow for superior predictive capabilities.

In addition, the methods and systems described herein provide for standard forms used by all employees of an IVF laboratory. The methods and systems document competency assessment records, time and date stamps results, and are completely confidential. These records become part of the laboratory's quality documents, and can be periodically reviewed and used for continuous improvement and quality assurance. Also, test pictures, videos, and written test questions are randomly refreshed every month from a large database of multimedia files to eliminate bias in a laboratory's QC testing procedures.

The invention, in one aspect, features a computerized method of assessing competency of an in vitro fertilization (IVF) technician. A computing device selects an image of a biological entity associated with an IVF procedure. The computing device executes one or more image analysis algorithms on the selected image to identify one or more morphological characteristics of the biological entity in the selected image. The computing device compares the identified morphological characteristics of the biological entity in the selected image with one or more morphological characteristics of a biological entity in a reference image. The computing device generates a similarity score between the biological entity in the selected image and the biological entity in the reference image based upon the comparison. The computing device determines an IVF procedure action for the biological entity in the selected image based upon the similarity score. The computing device requests input from a user at a client computing device relating to the biological entity in the selected image. The computing device determines whether the user input matches the IVF procedure action.

The invention, in another aspect, features a computerized method of automatically generating an in vitro fertilization (IVF) clinical recommendation. A computing device receives, from an image capture device, an image of a biological entity associated with an IVF procedure. The computing device executes one or more image analysis algorithms on the selected image to identify one or more morphological characteristics of the biological entity in the selected image. The computing device compares the identified morphological characteristics of the biological entity in the selected image with one or more morphological characteristics of a biological entity in a reference image. The computing device generates a similarity score between the biological entity in the selected image and the biological entity in the reference image based upon the comparison. The computing device determines a clinical recommendation for an IVF procedure that uses the biological entity in the selected image based upon the similarity score.

Any of the above aspects can include one or more of the following features. In some embodiments, the biological entity associated with the IVF procedure comprises an egg, a sperm cell, or an embryo. In some embodiments, the morphological characteristics comprise size, shape, cleavage, orientation, location, distance between sub-elements of the entity, structure, and length. In some embodiments, the image analysis algorithms include edge detection, noise suppression, image enhancement, pattern recognition, cell localization, and cell segmentation.

In some embodiments, comparing the identified morphological characteristics of the biological entity in the selected image with one or more morphological characteristics of a biological entity in a reference image comprises classifying, using an artificial intelligence image analysis algorithm executing on the computing device, the biological entity in the selected image as belonging to an image set that includes the reference image using the identified morphological characteristics. In some embodiments, the artificial intelligence image analysis algorithm comprises a neural network, a Random Forest algorithm, or an image classification modeling algorithm.

In some embodiments, determining an IVF procedure action for the biological entity in the selected image based upon the similarity score comprises determining whether the biological entity is suitable for inclusion in an IVF procedure based upon the similarity score, and when the biological entity is suitable for inclusion, identifying one or more IVF procedure actions for the biological entity based upon a type of the biological entity. In some embodiments, the computing device generates a user interface comprising the identified one or more IVF procedure actions and the selected image, for display to the user of the client computing device. In some embodiments, the input from the user of the client computing device is provided via the user interface and the input comprises a selection of one of the identified one or more IVF procedure actions.

In some embodiments, determining whether the user input matches the IVF procedure action comprises generating a competency score for the user based upon a comparison of the user input to the IVF procedure action. In some embodiments, the image capture device comprises a digital microscope. In some embodiments, the clinical recommendation comprises an action to be performed with respect to the biological entity as part of an IVF procedure

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the technology described above, together with further advantages, may be better understood by referring to the following description taken in conjunction with the accompanying drawings. The drawings are not necessarily to scale, emphasis instead generally being placed upon illustrating the principles of the technology.

FIG. 1 is a block diagram of a system for dynamic automation of quality control and information management for an IVF laboratory.

FIG. 2 is an exemplary flow diagram for configuration of the IVF software application for a Technologist/Clinical Staff user role.

FIG. 3 is a screenshot of an exemplary user interface of the IVF software application that depicts a plurality of different roles for which the application can be configured.

FIG. 4 is a screenshot of an exemplary user interface of the IVF software application that depicts a cryostorage inventory record for the patient, including image data.

FIG. 5 is a screenshot of an exemplary user interface of the IVF software application that depicts details about the IVF status for a particular patient.

FIG. 6 is a flow diagram of a computerized method for competency assessment of IVF technicians using artificial intelligence image analysis.

FIG. 7 is a screenshot of an exemplary user interface of the IVF software application that depicts a competency test question.

FIG. 8 is a flow diagram of a computerized method for biological entity assessment and validation during IVF using artificial intelligence image analysis.

FIG. 9 is a screenshot of an exemplary user interface of the IVF software application that depicts a recommended course of action

FIG. 10 is a screenshot of an exemplary user interface of the IVF software application that depicts a list of reports for a particular staff member.

FIG. 11 is a screenshot of an exemplary user interface of the IVF software application that depicts in-cycle statistics.

FIG. 12 is a screenshot of an exemplary user interface of the IVF software application that depicts a reagent use and expiration inventory.

### DETAILED DESCRIPTION

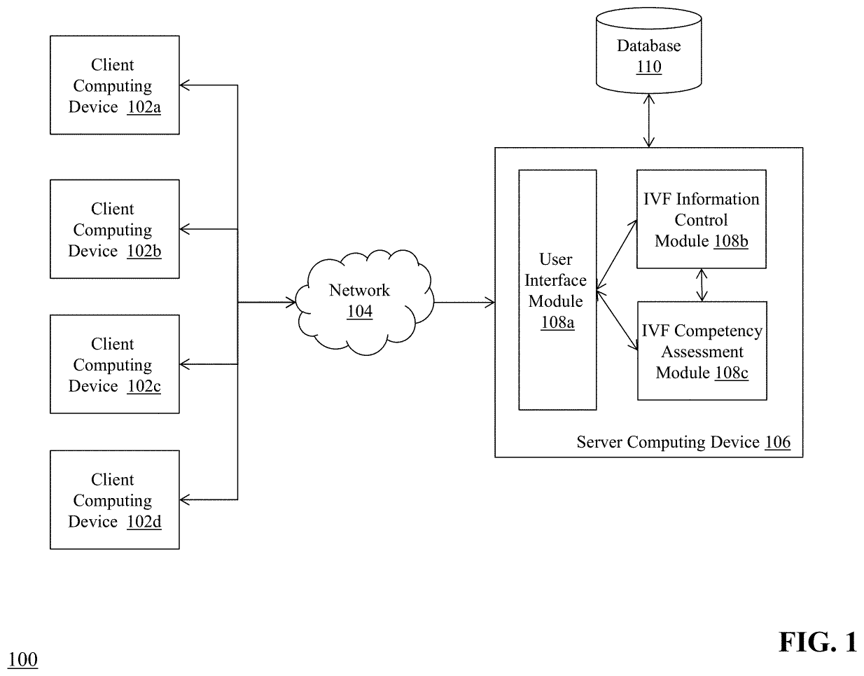

FIG. 1 is a block diagram of a system 100 for dynamic automation of quality control and information management for an IVF laboratory. The system of FIG. 1 includes a plurality of client computing devices 102a-102d, a communications network 104, a server computing device 106 with a user interface module 108a, an IVF information control module 108b, and an IVF competency assessment module 108c, and a database 110.

The client computing devices 102a-102d are connected to the communications network 104 in order to communicate with the server computing device 106 to provide input and receive output relating to the process of dynamic automation of quality control and information management for an IVF laboratory as described herein. In some embodiments, each client computing device 102a-102d may be coupled to a respective display device for, e.g., providing a detailed graphical user interface (GUI) that receives input for and presents output resulting from the methods and systems described herein. For example, the client computing device 102a-102d may connect to the user interface module 108a of server computing device 106, which provides, e.g., a web-based portal for users of the client computing devices 102a-102c to access functionality associated with the methods described herein.

Exemplary client devices 102a-102d include but are not limited to desktop computers, laptop computers, tablets, mobile devices, smartphones, and internet appliances. It should be appreciated that other types of computing devices that are capable of connecting to the components of the system of FIG. 1 may be used without departing from the scope of technology described herein. It also should be appreciated that each of the client computing devices 102a-102d may be associated with a different user type—for example, client computing device 102a may be associated with a patient accessing the system of FIG. 1 to generate a user profile and receive updates on an IVF process; client computing device 102b may be associated with a physician who is treating the patient and who accesses the system of FIG. 1 to analyze or update information for the patient; client computing device 102c may be associated with a director of an IVF laboratory or other facility that is administering the IVF process for the patient; and client computing device 102d may be associated with technologist or other staff member at the IVF laboratory.

The communications network 104 enables the other components of the system 100 to communicate with each other in order to conduct dynamic automation of quality control and information management for an IVF laboratory as described herein. The network 104 may be a local network, such as a LAN, or a wide area network, such as the Internet and/or a cellular network. In some embodiments, the network 104 is comprised of several discrete networks and/or sub-networks (e.g., cellular to Internet) that enable the components of the system of FIG. 1 to communicate with each other.

The server computing device 106 is a combination of hardware and software modules that includes specialized hardware and/or software modules which execute on a processor and interact with memory modules of the server computing device 106 to perform functions for dynamic automation of quality control and information management for an IVF laboratory as described herein. The server computing device 106 includes a user interface module 108a, an IVF information control module 108b, and an IVF competency assessment module 108c (as mentioned above) that execute on and/or interact with the processor of the server computing device 106.

In some embodiments, the user interface module 108a, the IVF information control module 108b, and the IVF competency assessment module 108c are specialized sets of computer software instructions programmed onto one or more dedicated processors in the server computing device 106 and may include specifically-designated memory locations and/or registers for executing the specialized computer software instructions. Although the modules 108a-108c are shown in FIG. 1 as executing within the same server computing device 106, in some embodiments the functionality of the modules 108a-108c may be distributed among a plurality of server computing devices. As shown in FIG. 1, the server computing device 106 enables the modules 108a-108c to communicate with each other in order to exchange data for the purposes of performing the described functions. It should be appreciated that any number of computing devices, arranged in a variety of architectures, resources, and configurations (e.g., cluster computing, virtual computing, cloud computing) may be used without departing from the scope of the technology described herein. The exemplary functionality of the modules 108a-108c is described in detail below.

The database 110 is a computing device (or in some embodiments, a set of computing devices) coupled to the server computing device 106 and is configured to receive, generate, and store specific segments of data relating to the process of dynamic automation of quality control and information management for an IVF laboratory as described herein. In some embodiments, all or a portion of the database 110 may be integrated with the server computing device 106 or be located on a separate computing device or devices. The database 110 may comprise one or more databases configured to store portions of data used by the other components of the system of FIG. 1, as will be described in greater detail below. In some embodiments, the database 110 comprises an enterprise business suite, such as Oracle E-Business Suite (EBS), that includes various modules that enable a spectrum of functionality to support the methods and systems described herein—including logistics, supply chain, transportation, CRM, and other types of modules. In another embodiment, the database 110 is MySQL™ available from Oracle Corp. of Redwood City, Calif.

Because the system 100 involves the storage of sensitive health-related information, the system 100 must be configured to comply with HIPAA guidelines. The server computing device 106 is configured to send data to the database 110 using, e.g., a Representational State Transfer (REST) application programming interface (API) developed with the Laravel framework (available at laravel.com). Protecting patient data at the point of transmission has been identified as a critical factor in HIPAA compliance, so the system 100 uses the “Passport authentication” method in Laravel to authenticate information, while avoiding security threats during data transfer. Statistical reports are also visible in the administrative panel, access to which is controlled by login and authentication that has also been developed in the Laravel framework. The database 110 is also protected with encryption techniques so that the raw values of the database cannot be decoded, including passwords.

In some embodiments, one or more of the client computing devices 102a-102d are mobile computing devices that execute one or more native applications and/or one or more browser applications for connecting to and interfacing with the server computing device 106. The native application is a software application (also called an ‘app’) that is installed locally on the mobile device 102 and written with programmatic code designed to interact with an operating system that is native to the mobile device 102. Such software is available from, e.g., the Apple® App Store or the Google® Play Store. In some embodiments, the native application 103a includes a software development kit (SDK) module that is executed by a processor of the mobile device 102 to perform functions associated with dynamic automation of quality control and information management for an IVF laboratory as described herein.

The browser application comprises software executing on a processor of the mobile device 102 that enables the mobile device to communicate via HTTP or HTTPS with remote servers addressable with URLs (e.g., sever computing device 106) to receive website-related content, including one or more webpages, for rendering in the browser application and presentation on a display device coupled to the mobile device 102. Exemplary mobile browser application software includes, but is not limited to, Firefox™, Chrome™, Safari™, and other similar software. The one or more webpages can comprise visual and audio content for display to and interaction with a user.

Either the native application or the browser application can be configured to present a user interface of the application on a display device coupled to the corresponding client computing device. For example, the client computing device 102a can connect to the user interface module 108a of server computing device 106 and during the communication session, the user interface module 108a can transmit data (e.g., code files, images, audio, etc.) that enable the client computing device 102a to render one or more user interface screens associated with a particular software application, such as the IVF quality control and information management software application described herein.

As can be appreciated, the system 100 can be configured to deploy role-based access management for the IVF application—where each user of a client computing device 102a-102d that accesses the server computing device 106 can be assigned one or more user roles (e.g., Technologist, Patient, Director, Administrator, Physician, etc.) that define the permissions and level of access afforded to that user by the server computing device 106 with respect to the functionality of the IVF software application. For example, a Technologist may have access to different modules (and sub-functions within those modules) within the IVF software application (e.g., testing, inventory, etc.) than a Patient would, and vice versa. These user roles can be data structures that are stored in database 110 and associated with one or more user- or client device-based credentials, so that when a particular user logs in to use the IVF software application with a set of user credentials (e.g., username, password), the server computing device 106 and/or the client computing device 102a-102d can authenticate the user credentials and select the appropriate role associated with the user credentials in order to initialize and configure the IVF software application for use.

In one embodiment, there can be at least five different types of user roles: Director, Technologist, Administrator, Physician, and Patient. Each of these roles can have certain permissions and functionality associated with it, such as:

Director: Initiate practice set-up, view all staff in a single dashboard, customize buttons or images for competency assessments, link reporting to various agencies, access, record and report pregnancy outcome data from web-based console or mobile app, track reagents and image certificates of analysis, create and add QC/QA reports and forms for staff members.

Technologist: Register under a laboratory director and practice, access assessments, receive and respond to messages via inbox, track procedures and success rates during training or on-boarding, compare assessment scores to technologists worldwide, view graphs and statistics for in-cycle parameters, record IVF cycle data for patients, report PGT results, track reagents in stock and in use.

Administrator: Human resource functions to control data during hiring and resignation periods, view and filter IVF practice statistics, view current and past patients and cycle data.

Physician: Single dashboard for current and past patients. View any image data (ultrasounds, eggs, cleavage stage embryos, etc.), cryostorage inventory, PGT results, patient reports, personal pregnancy outcome-related statistics.

Patient: Register demographic data, invite partner (can separate from partner record), change physician within network and automatically transfer records, view cryostorage inventory, PGT results, image data and any reports created by lab, patient satisfaction surveys.

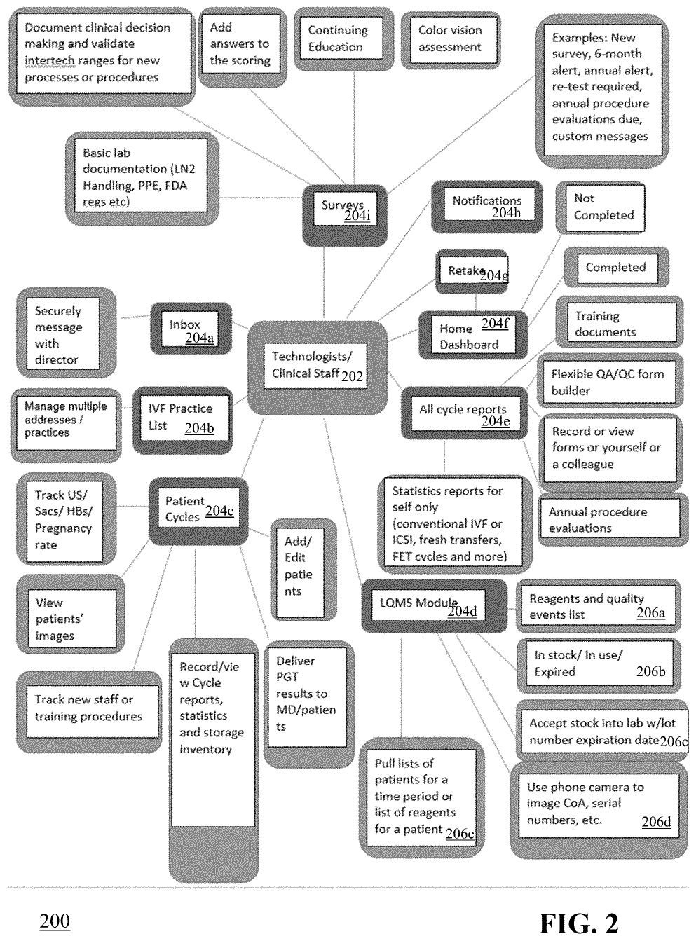

FIG. 2 is an exemplary flow diagram of a computerized method 200 for configuration of the IVF software application for a Technologist/Clinical Staff user role. As shown in FIG. 2, the root node 202 of the Technologist/Clinical Staff user role data structure is located at the center of the diagram, and the root node 202 is connected to a plurality of functions/modules available in the IVF software application to which the user role has access. In this example, the Technologist/Clinical Staff user role has access to a messaging inbox function 204a, an IVF Practice List function 204b, a Patient Cycles function 204c, a Laboratory Quality Management System (LQMS) module 204d, an All Cycle Reports module 204e, a Home Dashboard 204f, a Retake module 204g, a Notifications module 204h, and a Surveys module 204i. Furthermore, each software module or function 204a-204f provides one or more sub-functions or features within the corresponding module to which the Technologist/Clinical Staff user role has access. In one example, the LQMS module 204d provides the Technologist/Clinical Staff user role with the ability to View Reagents and Quality Events List 206a, View In Stock/In Use/Expired materials 206b, Accept Stock into Lab with Lot Number Expiration Date 206c, Use Phone Camera to Image CoA, Serial Numbers, etc. 206d, and Pull Lists of Patients for a Time Period for a List of Reagents for the Patent 206e. As can be understood, these exemplary functions enable the Technologist/Clinical Staff user role to efficiently analyze, review, and manage inventory and stock materials for the IVF Laboratory via the client computing device. As can be appreciated, other user roles may also have access to the same modules and sub-functions as shown in FIG. 2, and/or other user roles may have access to different modules and sub-functions, depending upon the specific requirements and permissions afforded to the user role. In addition, configuration of the IVF software application can be further refined to an individual user basis—where a user can have customized access to data and functionality in the IVF application.

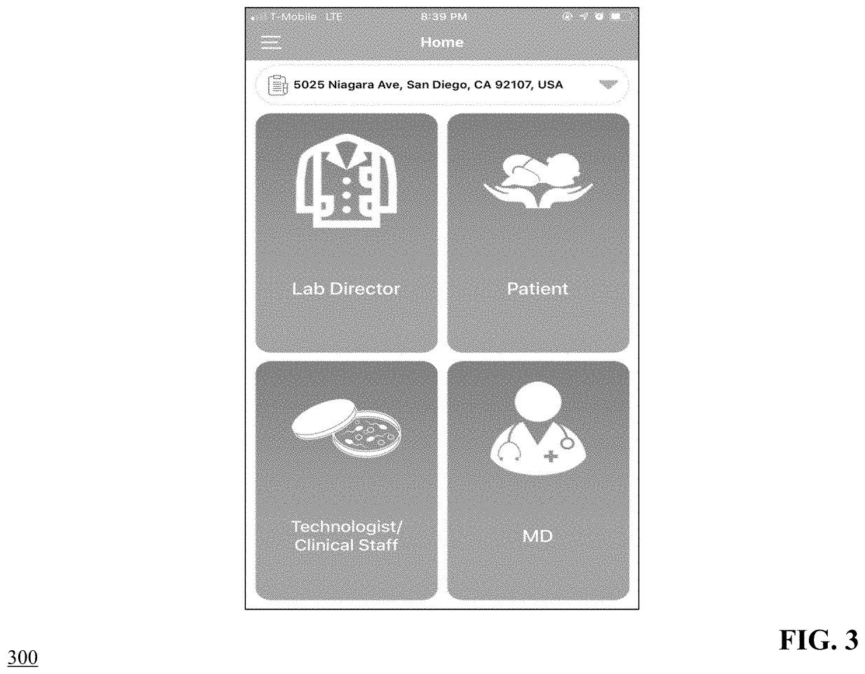

FIG. 3 is a screenshot of an exemplary user interface 300 of the IVF software application that depicts a plurality of different roles for which the application can be configured. As shown in FIG. 3, each role is assigned to a different are of the user interface and in some embodiments, the role can be selected (e.g., clicked or tapped) in order to configure the IVF application for that role.

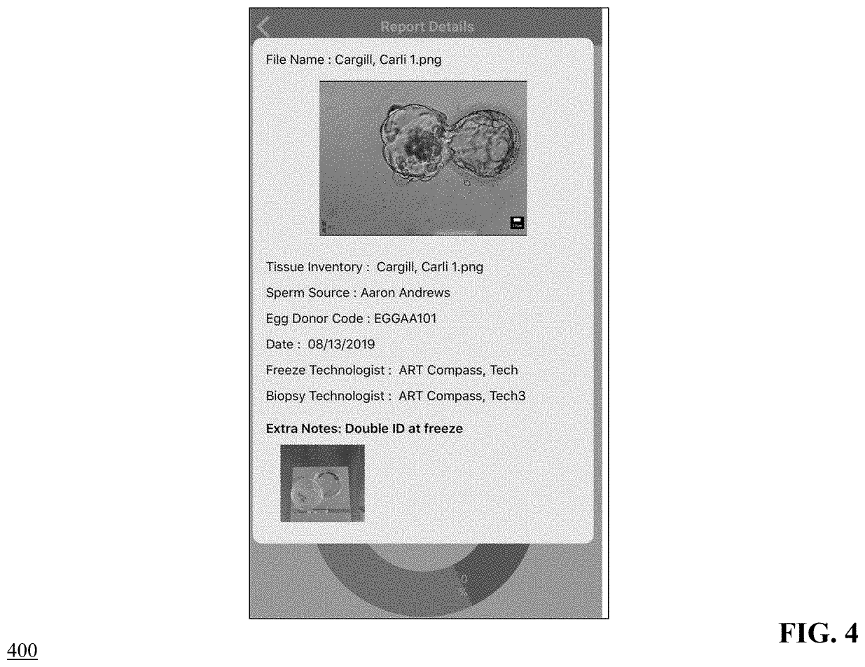

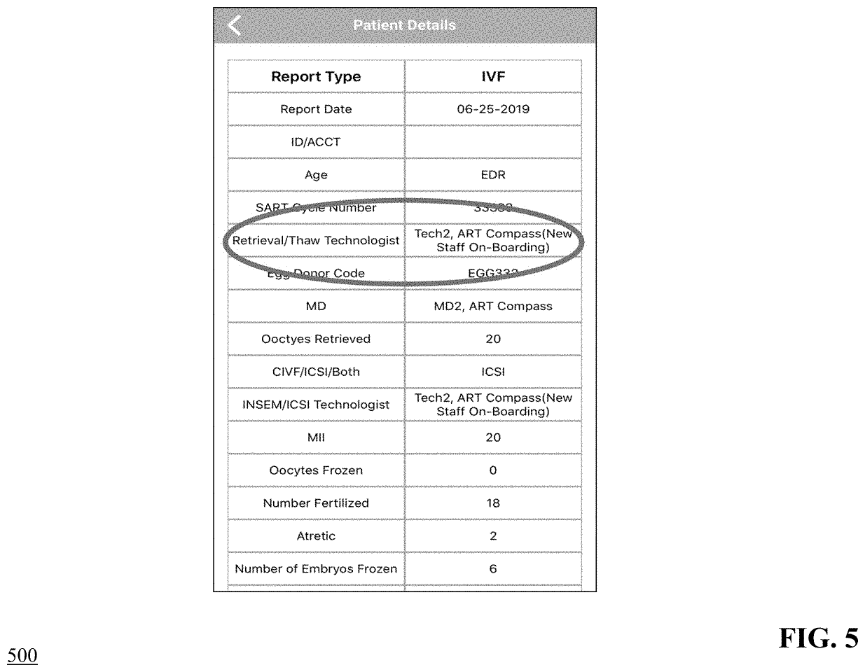

As mentioned above, a patient can be provided access to the IVF application in order to register demographic data, invite partner, change physician within network and automatically transfer records, view cryostorage inventory, PGT results, image data and any reports created by lab, and fill out patient satisfaction surveys. FIG. 4 is a screenshot of an exemplary user interface 400 of the IVF application that depicts a cryostorage inventory record for the patient, including image data. As shown in FIG. 4, the user interface provides a detailed view of a particular embryo of the patient in cryostorage. FIG. 5 is a screenshot of an exemplary user interface 500 of the IVF application that depicts details about the IVF status for a particular patient. As shown in FIG. 5, the user interface includes information about the technologist(s), physician (e.g., MD), as well as statistics relating to the inventory for the patient. This enables the patient to be better informed and in control of the IVF process.

Another important aspect of the methods and systems described herein is the evaluation of competency of personnel responsible for testing at the IVF Laboratory. Evaluating and documenting competency of personnel responsible for testing is typically required at least semiannually during the first year the individual tests patient specimens, and at least annually thereafter. Competency assessment must be performed for testing personnel for each test that the individual is approved by the laboratory director to perform. The following tables detail the procedures which are the minimal CLIA and WHO regulatory requirements for assessment of competency for all personnel performing laboratory testing, mapped to the corresponding functionality offered by the IVF software application to enable the IVF personnel to satisfy the CLIA requirements:

| | IVF Software Application |
|---|---|
| | Functionality |
| CLIA Requirements | |
| Direct observations of routine | Observation of Performance |
| patient test performance, | |
| including patient preparation, if | |
| applicable, specimen handling, | |
| processing and testing. | |
| Monitoring the recording and | Test Report Completion |
| reporting of test results | |
| Review of intermediate test results | QC/PT/PM Records |
| or worksheets, quality control | |
| records, proficiency testing | |
| results, and preventive | |
| maintenance records | |
| Direct observations of | Instrument Maintenance |
| performance of instrument | |
| maintenance and function checks | |
| Assessment of test performance | Peer Assessment |
| through testing previously | |
| analyzed specimens, internal blind | |
| testing samples or external | |
| proficiency testing samples | |
| Assessment of problem solving | Problem Solving |
| skills | |
| WHO Competency Assessment | |
| Procedure Recommendations | |
| The assessor contacts the | Push notifications, employee |
| employee in advance to inform | dashboard, application inbox. |
| her/him that the assessment will | |
| be done at a prearranged time. | |
| The assessment can be done while | Smart-phone design allows for |
| the employee is performing tasks | integration into daily work flow. |
| using routine sample images. | Multimedia image and video |
| | database eliminates bias and is |
| | more similar to routine sample |
| | analysis. |
| The assessment is done by a | Standardized test protocols and |
| specified method previously | check for understanding modules. |
| described and is recorded in a | A digital record is permanently |
| digital “logbook.” | saved to administrator console |
| | and displayed in director and |
| | technologist dashboards. |
| The results of the assessment are | Technologist dashboard |
| shared with the employee. | |
| A remedial action plan is | The app communicates specific |
| developed defining required | steps to be taken to correct the |
| retraining. | problem with related deadlines, |
| | date and time stamped, through |
| | the inbox. For example, the |
| | employee may need an updated |
| | version of the standard operating |
| | protocol (SOP). |
| The employee is asked to | Inbox records the interaction and |
| acknowledge the assessment, | response and date and time |
| related action plan, and | stamps it. Tests attempted, |
| reassessment. | completed and passed, or tests to |
| | be re-attempted are prominently |
| | displayed through badges on the |
| | technologist dashboard. |

As can be appreciated, competency can be evaluated in any of a variety of different performance aspects, including but not limited to: (i) competency in selection and evaluation of specimens (eggs, sperm) to be used in an IVF procedure; (ii) competency in performing a fertilization procedure and (iii) competency in analyzing embryos to determine suitability for implantation. Advantageously, the systems and methods described herein provide for artificial intelligence-based image analysis that enables the competency assessment and clinical decision-making as described herein.

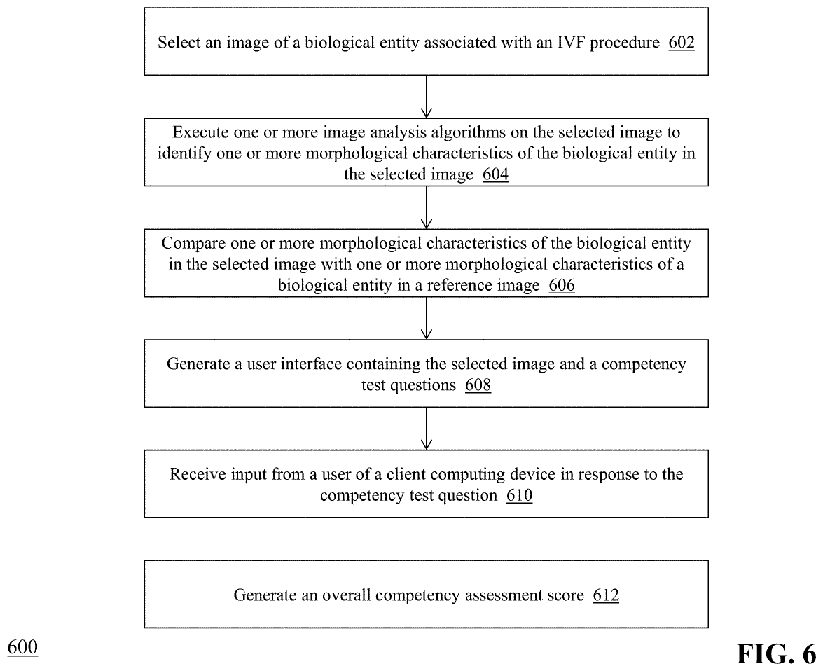

FIG. 6 is a flow diagram of a computerized method 600 for competency assessment of IVF technicians using artificial intelligence image analysis, using the system 100 of FIG. 1. A technician at client computing device 102a logs into the server computing device 106 and the user interface module 108a displays a competency assessment tool for the technician. Generally, the competency assessment tool displays images of eggs, sperm cells, and/or embryos at various stages of fertilization and growth and asks the technician to, e.g., make a clinical decision and/or provide a technical assessment of the entity presented in the image—in order to evaluate the technician's competency and compare his or her feedback with that of other technicians, etc. to gain a better understanding of the overall competency of the staff at a particular clinic or laboratory.

Continuing with FIG. 6, the IVF competency assessment module 108c selects (step 602) an image of a biological entity (e.g., egg, sperm cell, embryo) associated with an IVF procedure. The IVF competency assessment module 108c executes (step 604) one or more image analysis algorithms on the selected image to identify one or more morphological characteristics of the biological entity in the selected image. As can be appreciated, biological entities that are viable and/or preferable for inclusion in the IVF procedure exhibit certain morphological characteristics (such as shape, size, location, orientation, cleavage, structure, spatial relationship of sub-elements in the entity, etc.), whereas other biological entities that exhibit other morphological characteristics may be considered as less viable or not suitable for inclusion in the IVF procedure (due to, e.g., variation or abnormality in the above-referenced characteristics). Exemplary image analysis algorithms that the module 108c can apply to the selected image include, but are not limited to: edge detection, noise suppression, image enhancement, pattern recognition, cell localization, and cell segmentation. Exemplary algorithms for morphological analysis of biological entities in images are described in C. Cattani et al., “Biomedical Signal Processing and Modeling Complexity of Living Systems,” Computational and Mathematical Methods in Medicine, Volume 2012, doi: 10.1155/2012/298634; F. Ghasemian et al., “An efficient method for automatic morphological abnormality detection from human sperm images,” Comput. Methods Programs Biomed., December 2015, 122(3):409-20, doi: 10.1016/j.cmpb.2015.08.013; and E. Santos Filho et al., “A Review on Automatic Analysis of Human Embryo Microscope Images,” Open Biomed. Eng. J., 2010, 4:170-177, doi: 10.2174/1874120701004010170; each of which is incorporated herein by reference. The module 108c can automatically determine one or more morphological characteristics of the biological entit(ies) in the selected image using techniques such as those described above and store the determined morphological characteristics in database 110.

To determine the viability or suitability of biological entities in the image, the module 108c compares (step 606) one or more morphological characteristics of the biological entities in the image with morphological characteristics of biological entities depicted in one or more images in a reference data set stored in database 110. For example, the database 110 can be configured to store images of reference biological entities and/or associated morphological characteristics of said biological entities in the images. The reference biological entities can correspond to successful IVF procedures—e.g., the entities have morphological characteristics that are highly predictive of a successful IVF procedure. The module 108c can compare one or more morphological characteristics of the entity in the selected image with one or more morphological characteristics of the entity in the reference image to assess how close or divergent the entity in the selected image is from the entity in the reference image—which can be indicative of how viable or suitable the entity in the selected image is for IVF. In some embodiments, the module 108c applies a weighting algorithm to one or more of the morphological characteristics depending upon, e.g., the importance that certain characteristics have to overall likelihood of successful IVF/implantation. For example, the shape of a sperm cell head may have a stronger correlation to successful fertilization and embryo viability than tail length—as a result, the module 108c can weigh the shape comparison result more heavily than tail length comparison when evaluating the suitability of a sperm cell. In some embodiments, the module 108c can generate a suitability score for the biological entity represented in the selected image, where the score indicates the viability of the biological entity for inclusion in the IVF procedure (e.g., a scale of 1-100, where 1 is low viability and 100 is high viability).

In some embodiments, the IVF competency assessment module 108c can utilize one or more artificial intelligence (AI)-based image analysis methods for conducting the comparison between the selected image and the reference image(s). For example, the module 108c can be configured to execute a convolutional neural network (CNN), a Random Forest algorithm, an image classification model, and other types of AI algorithms that analyze one or more features (e.g. morphological characteristics) of the biological entities in the selected image to classify the entities as viable or non-viable. Exemplary AI algorithms that can be used by the module 108c are described in P. Khosravi et al., “Deep learning enables robust assessment and selection of human blastocysts after in vitro fertilization,” npj Digital Medicine 2, Article No. 21 (2019), doi: 10.1038/s41746-019-0096-y; M. VerMilyea et al., “Development of an artificial intelligence-based assessment model for prediction of embryo viability using static images captured by optical light microscopy during IVF,” Human Reproduction, Vol. 35, Issue 4, April 2020, pp. 770-784; and C. McCallum et al., “Deep learning-based selection of human sperm with high DNA integrity,” Communications Biology 2, Article No. 250 (2019), doi: 10.1038/s42003-019-0491-6, all of which are incorporated herein by reference. The IVF competency assessment module 108c can classify the biological entities depicted in one or more selected images using the above-referenced AI techniques to provide an automatic assessment of the viability and/or suitability of the biological entities for the IVF process—whereas entities that have characteristics that more closely resemble characteristics of similar entities that were used in known successful IVF procedures can be classified as ‘suitable,’ and entities that do not have such characteristics can be classified as ‘unsuitable.’ One of skill in the art can readily appreciate that the above-referenced techniques are merely exemplary, and that other types of image analysis techniques can be used without departing from the scope of invention described herein.

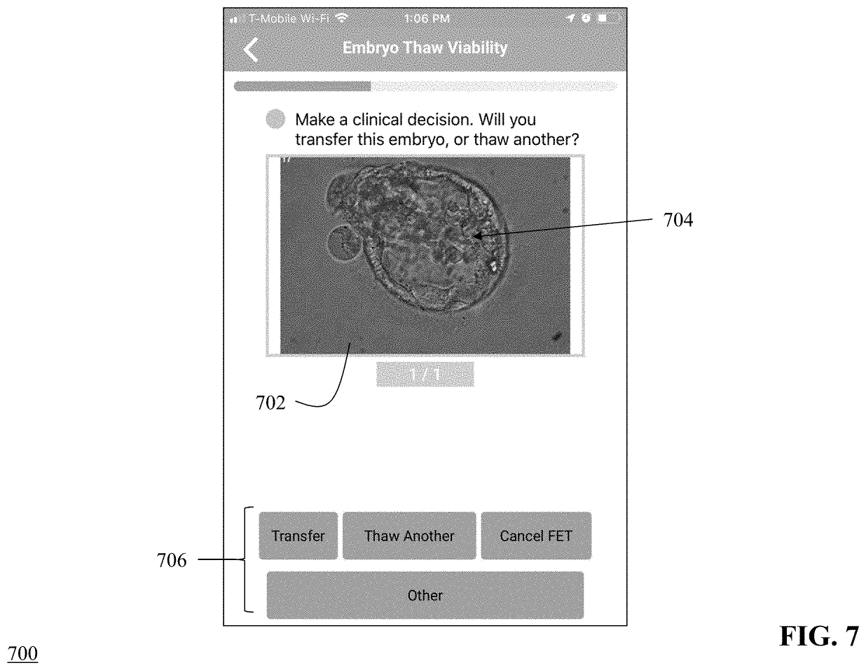

After classification of the biological entities in the selected image and/or comparison of the morphological characteristics of the biological entities to reference characteristics as described above, the module 108c generates a user interface (step 608) containing the selected image of the biological entities to the technician at client computing device 102a via the UI module 108a. The user interface also contains a competency test question and/or decision point for the technician's review as relating to the biological entities in the displayed image, along with user interface elements that provide the technician with a means to submit input (step 610) in response to the decision point. FIG. 7 is a screenshot of an exemplary user interface 700 of the IVF application that depicts a competency test question. As shown in FIG. 7, the user interface 700 displays an image 702 of a biological entity 704 (in this case, an embryo that is a candidate for transfer) and asks the user to make a clinical decision as to whether to transfer the embryo or thaw another. The user can select one of the option buttons 706 below the image with his or her answer.

As described above, the module 108c has already analyzed the image 702 presented in the user interface 700 based upon, e.g., the morphological characteristics of the embryo 704 to classify the embryo as ‘suitable’ or ‘unsuitable’ for IVF, prior to presenting the image to the technician. A competent technician should be able to analyze the visual characteristics of the embryo 704 in image 702 and correctly answer the question presented, i.e., whether to transfer this embryo (because it displays morphological characteristics that compare favorably to successful embryos and therefore make it a good candidate for transfer) or to thaw another (because this embryo displays morphological characteristics that are undesirable for a successful IVF procedure). When the technician's answer aligns with the assessment of the IVF competency assessment module 108c, the module 108c marks the answer as correct and proceeds onto the next question—where an answer that does not align with the module's 108c assessment is marked as incorrect.

The module 108c can present any number of test questions/decision points for the technician's review and upon completion of the competency assessment, the module 108c generates (step 612) an overall competency assessment score for the technician. In some embodiments, the module 108c enables the technician to review his or her answers and determine areas for further testing and/or improvement. The module 108c can also store the technician's score in database 110 for future analysis (such as comparison with other technicians' scores in the same lab or other labs, comparison with the same technician's past or future scores, etc.). In this way, the system 100 provides for an automatic and dynamic competency assessment application for IVF technicians, which promotes better efficiency and success rates when carrying out actual IVF clinical decision-making.

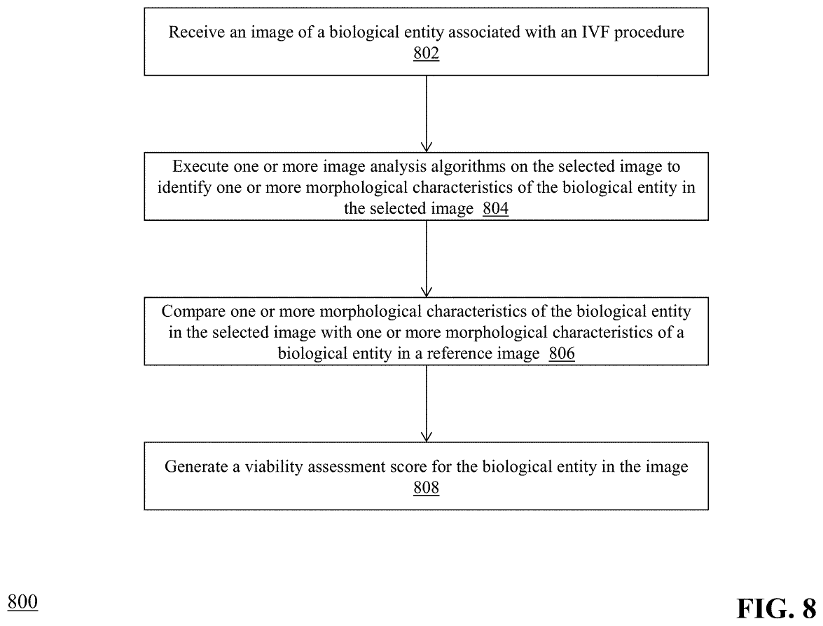

The above-referenced image analysis techniques are useful not only for competency assessment, but also for automatically identifying suitable biological entities during IVF for patients. FIG. 8 is a flow diagram of a computerized method 800 for biological entity assessment and validation during IVF using artificial intelligence image analysis, using the system 100 of FIG. 1. A technician can capture one or more images of a biological entity that is a candidate for use during an IVF procedure. For example, the technician may be selecting specimens of sperm cells or eggs to be used in fertilization, or the technician may be evaluating the suitability of an embryo for implantation. The technician can analyze the specimen(s) under a microscope (such as the Zeiss Axiolab 5, Zeiss Stemi 508, or Zeiss Axio Observer, available from Zeiss Group of Oberkochen, Germany) with functionality that enables capture of digital images of the biological entities under examination. The microscope can transfer the images to the server computing device 106, where the IVF information control module 108b receives (step 802) the images from the microscope.

The IVF information control module 108b executes (step 804) one or more image analysis algorithms on the received image(s) to determine one or more morphological characteristics of the biological entities in the image(s). In some embodiments, the module 108b uses the same or similar image analysis algorithms as the IVF competency assessment module 108c for this purpose, such as edge detection, noise suppression, image enhancement, pattern recognition, cell localization, and cell segmentation (see, e.g., Cattani, Ghasemian, Santos Filha, supra).

After determining the morphological characteristics of the biological entities, the IVF information control module 108b conducts (step 806) a comparison of the morphological characteristics with morphological characteristics of biological entities depicted in one or more images in a reference data set stored in database 110. In some embodiments, the module 108b uses the same or similar image analysis algorithms as the IVF competency assessment module 108c for this purpose, such as weighting algorithms, CNNs, Random Forest, image classification models, and other types of AI algorithms (see, e.g., Khosravi, VerMilyea, McCallum, supra).

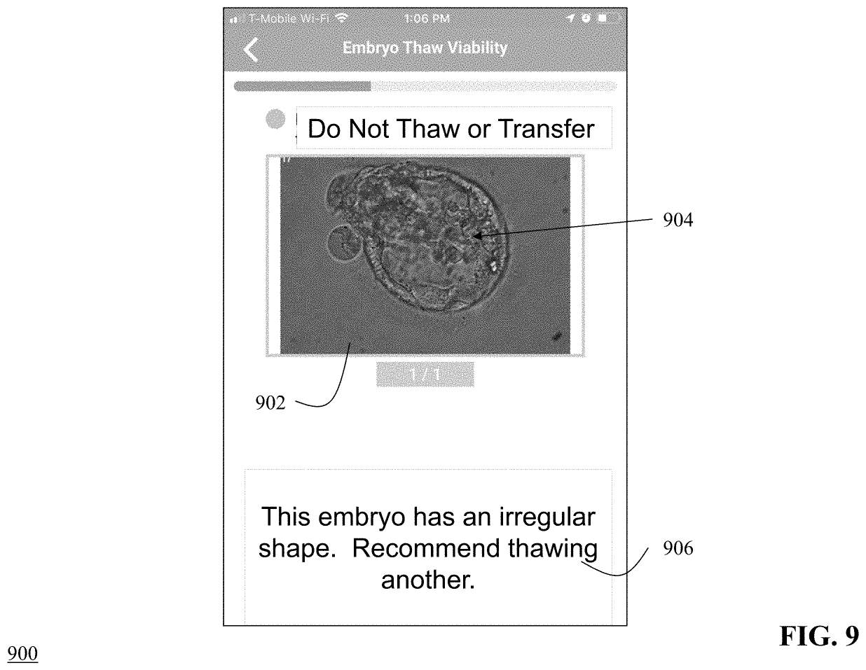

The module 108b then generates (step 808) a viability assessment score for the biological entities in the captured image(s) based upon the comparison step above. For example, the module 108b can determine that, due to the divergence of its morphological characteristics from an accepted reference set, a sperm cell in a captured image is not suitable for fertilization. The module 108b can generate a user interface for display to the technician at client computing device 102a that comprises the digital image of biological entity under observation in the microscope, and the recommended course of action for the technician's review (e.g., “Do Not Thaw”). FIG. 9 is a screenshot of an exemplary user interface 900 of the IVF application that depicts a recommended course of action. As shown in FIG. 9, the user interface 900 comprises the image 902 of an embryo 904 as captured from the microscope. The user interface 900 also comprises a notification message 906 for the technician, indicating the recommended course of action based upon the image analysis and comparison performed by the module 108b.

Other advantageous features of the methods and systems for dynamic automation of quality control and information management for an IVF laboratory as described herein include the following:

Validation Studies—over eighty competency assessment modules have been created and made available in the IVF software for sperm and embryo morphology, quality, viability and common clinical decision timepoints. Validation studies are underway, using images of PAP stained morphology slides for andrology modules, and 112 cleavage stage images captured on EmbryoScope 66 hours post insemination, and 168 blastocyst stage images captured on EmbryoScope 115 and 139 hours post insemination for embryology modules. Each slide was rotated and repeated 3 times throughout the modules to allow for intra-observer variability measurements.

Test Protocol Standardization and Check for Understanding—Rigorously standardized test protocols are essential for meaningful comparisons across multiple sites. Before each assessment can begin, the IVF software displays a standardized and detailed protocol, followed by a quick “Check for Understanding” module to ensure that test takers understand the instructions and how to take the test. The IVF software application is flexible; it can serve standardized specimens to each technologist at each study site simultaneously, allowing even very small IVF clinics to compare an individual values to the mean of all technologists.

Statistics—The IVF software application beneficially standardizes the analysis of QC data in accordance with published standards. For example, when calculating the coefficients of variation, some investigators use raw data, some transform the means, others do not perform any type of statistical analysis at all. The IVF software application provides instant comparative results generation logic with statistical analysis and tracking tools to quantify inter- and intra-technician variability.

Clinical Decision Making—Embryologists rely on morphological assessments when assessing sperm morphology, sperm quality and when performing semen analyses and when selecting sperm for ICSI. Once a sperm has been identified for injection, embryologists immobilize the cell by breaking the tail with an ICSI needle. Breaking the cell membrane invokes subsequent physiological and biochemical reactions, which promotes decondensation of the sperm head and activation of the oocyte. Staff are trained to avoid contact with the midpiece region of the sperm as this contains the centriole, which plays a major role in the cleavage patterns of the developing embryo. The IVF software application described herein can advantageously render images of the cell in order to enable staff to visually determine the location of the immobilization step.

Additionally, cleavage stage embryo morphological assessments are performed to evaluate embryo quality. These scores are used to make clinical decisions such as determining the day of transfer (Day 3 vs Day 5), determining the number of embryos to transfer, selecting the top-quality embryo(s) for transfer, as well as determining the final disposition of embryos. The IVF software application provides assessments for each of these parameters.

Blastocyst stage embryo morphological assessments are performed to evaluate embryo quality. These scores are used to make clinical decisions regarding the number of embryos to transfer, selecting the top-quality embryo(s) for transfer, as well as determining the final disposition of embryos. The IVF software application provides assessments for each of these parameters.

New Staff On-boarding and Training—When new staff is hired, whether they are a senior level technologist with many years of training or a junior technologist new to the field, they must document the success rates and numbers of procedures performed during the training period. The IVF software application is configured to provide documentation of new staff on-boarding for LQMS records—including metrics such as percent of retrieval with a missed oocyte, ICSI-2pn, -atretic, -cleaving, and -usable blastocyst rate, embryo thaw survival, embryos biopsied and cells tubed no result/indeterminate result, embryo transfer positive, negative, and biochemical rate and more. Standard training reports provided by the IVF software application include; oocyte retrieval, vitrification and thaw, embryo thaw, vitrification, and transfer, ICSI, biopsy, andrology and more.

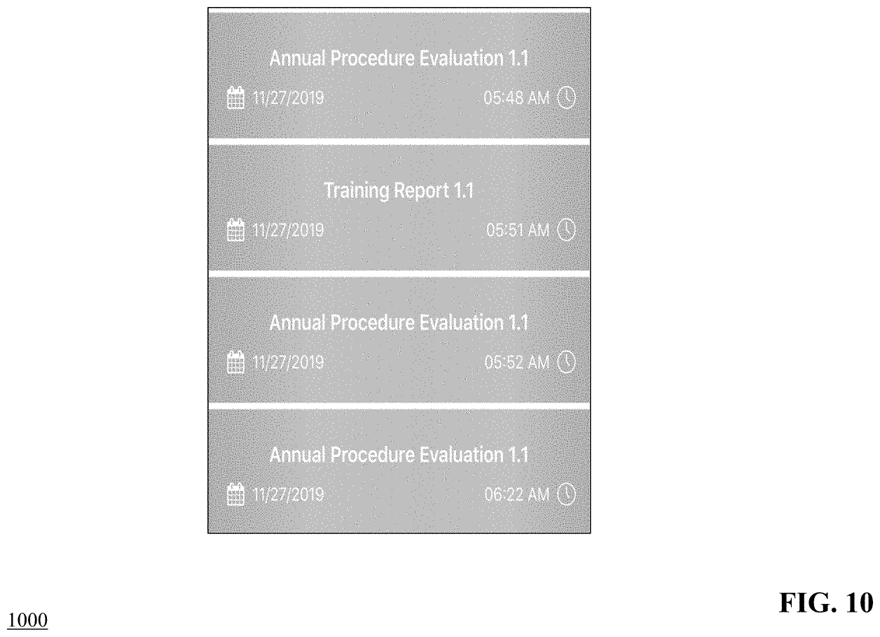

FIG. 7 is a screenshot of an exemplary user interface of the IVF software application that depicts a list of reports for a particular staff member. As shown in FIG. 10, the user interface includes a list of reports and evaluations associated with a particular staff member; interacting with one of the reports can provide additional detail on the evaluation or training report (e.g., scoring).

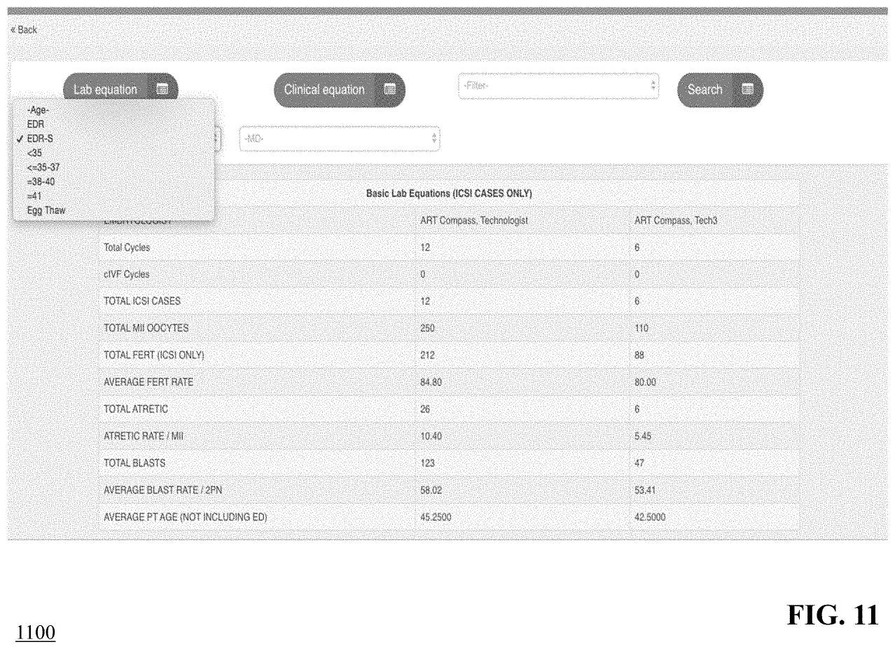

“In-Cycle” Statistics Module—in addition to the above features, the IVF software application is a versatile quality control tool to oversee large or small teams, high volumes of work, data collection, auditing, and accountability—and the in-cycle statistics module provides an additional level of performance monitoring. Key performance indicators have been established for clinical embryologists for use in monitoring ‘fresh’ IVF and ICSI cycles. They provide the basis for the quantitative performance criteria used by the IVF software application to create competency profiles. The in-cycle statistics module allows individual performance records within a clinic to be analyzed and compared to each other. FIG. 11 is a screenshot of an exemplary user interface of the IVF software application that depicts in-cycle statistics.

REST API for EMR Integration—the IVF software application further leverages a strong, flexible, and secure REST API to reduce “double documentation” by importing cycle statistics directly from, or exporting data to an electronic medical record (EMR) system.

Automated Reporting—the IVF software application has been designed to automate reporting by preparing system wide reports for various supervising bodies. Advance scheduling, automated reminders, and compliance tracking improves the compliance rate by both programs and technologists while preserving administrative staff time for other tasks. The Laboratory director, physicians and reproductive endocrinologists can compare performance across programs, and spot quality issues in a manner that allows early intervention.

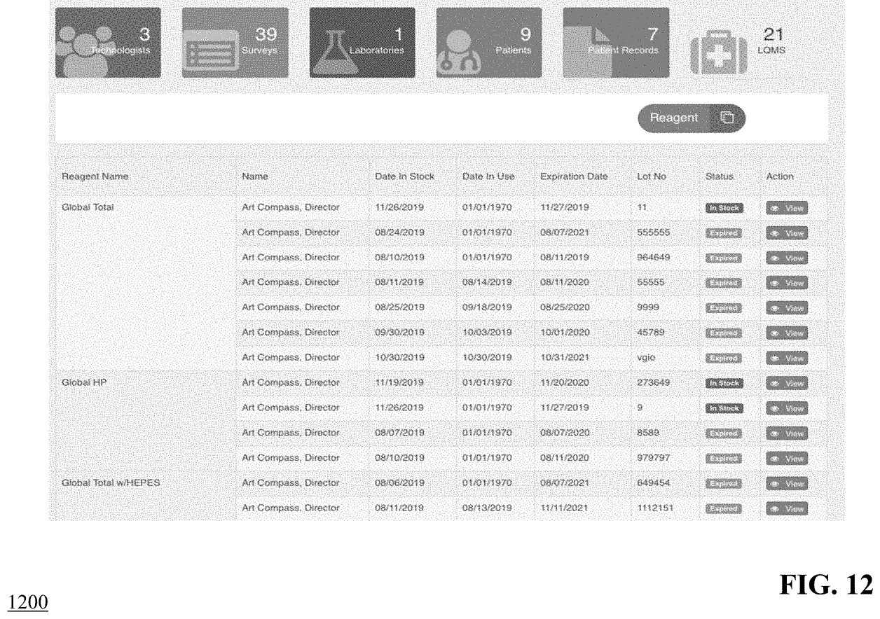

LQMS Module—as mentioned above, the IVF software application comprises a LQMS module that enables users to create lists of reagents and quality events, accept reagents into the lab stock (date received, lot number, expiration date), put them in to use, and expire them, search for all patients in a time period, or by patient ID to generate lists of reagents in use or quality events, use a device camera to image the certificate of analysis, the condition of the box, serial number on gas tanks, etc. FIG. 12 is a screenshot of an exemplary user interface of the IVF software application that depicts a reagent use and expiration inventory.

Method steps can be performed by one or more special-purpose processors executing a computer program to perform functions of the technology by operating on input data and/or generating output data. Method steps can also be performed by, and an apparatus can be implemented as, special-purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines can refer to portions of the stored computer program and/or the processor, and/or the special circuitry that implement one or more functions.

Processors suitable for the execution of a computer program include, by way of example, special-purpose microprocessors. Generally, a processor receives instructions and data from a read-only memory or a random access memory or both. The essential elements of a computer are a specialized processor for executing instructions and one or more specifically-allocated memory devices for storing instructions and/or data. Memory devices, such as a cache, can be used to temporarily store data. Memory devices can also be used for long-term data storage. Generally, a computer also includes, or is operatively coupled to receive data from or transfer data to, or both, one or more mass storage devices for storing data, e.g., magnetic, magneto-optical disks, or optical disks. A computer can also be operatively coupled to a communications network in order to receive instructions and/or data from the network and/or to transfer instructions and/or data to the network. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory can be supplemented by and/or incorporated in special purpose logic circuitry.

To provide for interaction with a user, the above described techniques can be implemented on a computing device in communication with a display device, e.g., a CRT (cathode ray tube), plasma, or LCD (liquid crystal display) monitor, a mobile device display or screen, a holographic device and/or projector, for displaying information to the user and a keyboard and a pointing device, e.g., a mouse, a trackball, a touchpad, or a motion sensor, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, and/or tactile input.

The above-described techniques can be implemented in a distributed computing system that includes a back-end component. The back-end component can, for example, be a data server, a middleware component, and/or an application server. The above described techniques can be implemented in a distributed computing system that includes a front-end component. The front-end component can, for example, be a client computer having a graphical user interface, a Web browser through which a user can interact with an example implementation, and/or other graphical user interfaces for a transmitting device. The above described techniques can be implemented in a distributed computing system that includes any combination of such back-end, middleware, or front-end components.

The components of the computing system can be interconnected by transmission medium, which can include any form or medium of digital or analog data communication (e.g., a communication network). Transmission medium can include one or more packet-based networks and/or one or more circuit-based networks in any configuration. Packet-based networks can include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks can include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Information transfer over transmission medium can be based on one or more communication protocols. Communication protocols can include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

Devices of the computing system can include, for example, a computer, a computer with a browser device, a telephone, an IP phone, a mobile device (e.g., cellular phone, personal digital assistant (PDA) device, smart phone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Microsoft® Internet Explorer® available from Microsoft Corporation, and/or Mozilla® Firefox available from Mozilla Corporation). Mobile computing device include, for example, a Blackberry® from Research in Motion, an iPhone® from Apple Corporation, and/or an Android™-based device such as the Pixel™ from Google Inc. IP phones include, for example, a Cisco® Unified IP Phone 7985G and/or a Cisco® Unified Wireless Phone 7920 available from Cisco Systems, Inc.

Comprise, include, and/or plural forms of each are open ended and include the listed parts and can include additional parts that are not listed. And/or is open ended and includes one or more of the listed parts and combinations of the listed parts.

One skilled in the art will realize the invention may be embodied in other specific forms without departing from the spirit or essential characteristics thereof. The foregoing embodiments are therefore to be considered in all respects illustrative rather than limiting of the invention described herein.

What is claimed is:

1. A computerized method of assessing competency of an in vitro fertilization (IVF) technician, the method comprising:
selecting, by a computing device, an image of a biological entity associated with an IVF procedure;
executing, by the computing device, one or more image analysis algorithms on the selected image to identify one or more morphological characteristics of the biological entity in the selected image;
comparing, by the computing device, the identified morphological characteristics of the biological entity in the selected image with one or more morphological characteristics of a biological entity in a reference image;
generating, by the computing device, a similarity score between the biological entity in the selected image and the biological entity in the reference image based upon the comparison;
determining, by the computing device, an IVF procedure action for the biological entity in the selected image based upon the similarity score;
requesting, by the computing device, input from a user at a client computing device relating to the biological entity in the selected image; and
determining, by the computing device, whether the user input matches the IVF procedure action.

2. The method of claim 1, wherein the biological entity associated with the IVF procedure comprises an egg, a sperm cell, or an embryo.

3. The method of claim 1, wherein the morphological characteristics comprise size, shape, cleavage, orientation, location, distance between sub-elements of the entity, structure, and length.

4. The method of claim 3, wherein the image analysis algorithms include edge detection, noise suppression, image enhancement, pattern recognition, cell localization, and cell segmentation.

5. The method of claim 1, wherein comparing the identified morphological characteristics of the biological entity in the selected image with one or more morphological characteristics of a biological entity in a reference image comprises classifying, using an artificial intelligence image analysis algorithm executing on the computing device, the biological entity in the selected image as belonging to an image set that includes the reference image using the identified morphological characteristics.

6. The method of claim 5, wherein the artificial intelligence image analysis algorithm comprises a neural network, a Random Forest algorithm, or an image classification modeling algorithm.

7. The method of claim 1, wherein determining an IVF procedure action for the biological entity in the selected image based upon the similarity score comprises:
determining whether the biological entity is suitable for inclusion in an IVF procedure based upon the similarity score; and
when the biological entity is suitable for inclusion, identifying one or more IVF procedure actions for the biological entity based upon a type of the biological entity.

8. The method of claim 7, wherein the computing device generates a user interface comprising the identified one or more IVF procedure actions and the selected image, for display to the user of the client computing device.

9. The method of claim 8, wherein the input from the user of the client computing device is provided via the user interface and the input comprises a selection of one of the identified one or more IVF procedure actions.

10. The method of claim 1, wherein determining whether the user input matches the IVF procedure action comprises generating a competency score for the user based upon a comparison of the user input to the IVF procedure action.

11. A computerized method of automatically generating an in vitro fertilization (IVF) clinical recommendation, the method comprising:
receiving, by a computing device from an image capture device, an image of a biological entity associated with an IVF procedure;
executing, by the computing device, one or more image analysis algorithms on the selected image to identify one or more morphological characteristics of the biological entity in the selected image;
comparing, by the computing device, the identified morphological characteristics of the biological entity in the selected image with one or more morphological characteristics of a biological entity in a reference image;
generating, by the computing device, a similarity score between the biological entity in the selected image and the biological entity in the reference image based upon the comparison; and
determining, by the computing device, a clinical recommendation for an IVF procedure that uses the biological entity in the selected image based upon the similarity score.

12. The method of claim 11, wherein the image capture device comprises a digital microscope.

13. The method of claim 11, wherein the biological entity associated with the IVF procedure comprises an egg, a sperm cell, or an embryo.

14. The method of claim 11, wherein the morphological characteristics comprise size, shape, cleavage, orientation, location, distance between sub-elements of the entity, structure, and length.

15. The method of claim 14, wherein the image analysis algorithms include edge detection, noise suppression, image enhancement, pattern recognition, cell localization, and cell segmentation.

16. The method of claim 11, wherein comparing the identified morphological characteristics of the biological entity in the selected image with one or more morphological characteristics of a biological entity in a reference image comprises classifying, using an artificial intelligence image analysis algorithm executing on the computing device, the biological entity in the selected image as belonging to an image set that includes the reference image using the identified morphological characteristics.

17. The method of claim 16, wherein the artificial intelligence image analysis algorithm comprises a neural network, a Random Forest algorithm, or an image classification modeling algorithm.

18. The method of claim 11, wherein the clinical recommendation comprises an action to be performed with respect to the biological entity as part of an IVF procedure.
